(19) United States
(12) Patent Application Publication
(10) Pub. No.: US 2009/0198183 A1
(43) Pub. Date: Aug. 6, 2009

(54) APPARATUS AND METHODS FOR INJECTING DERMAL FILLERS

(76) Inventors: John F. Krumme, Woodside, CA (US); John W. Parissenti, San Jose, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/187,964

(22) Filed: Aug. 7, 2008

Related U.S. Application Data
(63) Continuation-in-part of application No. PCT/US2007/023226, filed on Nov. 1, 2007.
(60) Provisional application No. 60/856,430, filed on Nov. 3, 2006.
(60) Provisional application No. 60/857,546, filed on Nov. 8, 2006.
(60) Provisional application No. 60/857,755, filed on Nov. 8, 2006.
(60) Provisional application No. 60/964,066, filed on Aug. 8, 2007.
(60) Provisional application No. 60/993,541, filed on Sep. 12, 2007.
(60) Provisional application No. 61/016,223, filed on Dec. 21, 2007.

(51) Int. Cl. A61M 5/315; A61M 5/20; B23P 11/00

(52) U.S. Cl. 604/143; 604/218; 29/428

(57) ABSTRACT

An apparatus includes a tubular member and a spherical piston disposed within a lumen defined by the tubular member. The tubular member includes a distal end portion and a central portion. The distal end portion is configured to be coupled to a needle. At least the central portion of the tubular member is curved. The spherical piston is movably disposed with the lumen such that the lumen is divided into a first portion and a second portion. The first portion of the lumen configured to contain a medicament.

### CROSS-REFERENCE TO RELATED APPLICATIONS

This application is a continuation-in-part of International Patent Application No. PCT/US2007/023226, entitled “Compositions, Devices and Methods for Modifying Soft Tissue,” filed Nov. 1, 2007, which claims priority to U.S. Provisional Application Ser. No. 60/856,430, entitled “Soft Tissue Modification,” filed Nov. 3, 2006, U.S. Provisional Application Ser. No. 60/857,546, entitled “Soft Tissue Modification,” filed Nov. 8, 2006, and U.S. Provisional Application Ser. No. 60/857,755, entitled “Injection Device,” filed Nov. 8, 2006, each of which is incorporated herein by reference in its entirety.

This application claims priority to U.S. Provisional Application Ser. No. 60/964,066, entitled “Controlled Injection Device,” filed Aug. 8, 2007, which is incorporated herein by reference in its entirety. This application claims priority to U.S. Provisional Application Ser. No. 60/993,541, entitled “Controlled Injection Device,” filed Sep. 12, 2007, which is incorporated herein by reference in its entirety. This application claims priority to U.S. Provisional Application Ser. No. 61/016,223, entitled “Self-Contained Pressurized Injection Device,” filed Dec. 21, 2007, which is incorporated herein by reference in its entirety.

### BACKGROUND

The invention relates generally to medical devices and methods for injecting dermal fillers into a body, and methods for producing pre-filled medicament containers.

Dermal fillers can be injected into the body to augment soft tissue portions of the body. For example, known dermal fillers can be injected adjacent the urinary sphincter muscle to increase the volume of the tissue within the urinary tract to treat urinary incontinence. Dermal fillers can also be injected into the skin and/or beneath the skin to change the contour of and/or increase the volume of the skin. For example, known dermal fillers can be injected within facial skin to remove wrinkles, treat scars or the like. Known dermal fillers can also be injected into and/or beneath the skin for breast augmentation.

Some known procedures for injecting dermal fillers include injecting the dermal filler using a known syringe that is filled with a predetermined amount of the dermal filler (e.g., one cubic centimeter). Such known syringes are often pre-filled in a sterile environment, and thus are often available only in a limited number of sizes as dictated by the manufacturer. The size can be limited, for example, as a function the user's hand strength in dispensing the dermal filler from the syringe at a desired pressure. For example, some known syringes are available only in sizes that contain one or two cubic centimeters of dermal filler. Accordingly, for procedures in which a higher volume of dermal filler is desired (e.g., breast augmentation), a user may be required to make multiple injections using multiple syringes to inject the desired volume of dermal filler. Alternatively, the user may inject less than the desired volume of dermal filler.

Moreover, such known syringes are constructed of rigid materials, are cylindrical in shape, and can include a plunger extending from the proximal end for generating the injection pressure to inject the dermal filler. Thus, during some known procedures, the orientation and/or positioning of the syringe can be limited by the size and/or length of the syringe. Similarly stated, during some known procedures, a user may not be able to position the needle of the syringe as desired because a portion of the syringe interferes the patient's body and/or other structure adjacent the syringe.

Thus, a need exists for improved apparatus and methods for injecting dermal fillers into a body. Moreover, a need exists for improved methods for producing pre-filled medicament containers.

### SUMMARY

Apparatus and methods of injecting dermal fillers are described herein. In some embodiments, an apparatus includes a tubular member and a spherical piston disposed within a lumen defined by the tubular member. The tubular member includes a distal end portion and a central portion. The distal end portion is configured to be coupled to a needle. At least the central portion of the tubular member is curved. The spherical piston is movably disposed with the lumen such that the lumen is divided into a first portion and a second portion. The first portion of the lumen configured to contain a medicament.

### BRIEF DESCRIPTION OF THE DRAWINGS

FIGS. 1 and 2 are schematic illustrations showing a medical injector according to an embodiment in a first configuration and a second configuration, respectively.

FIGS. 3 and 4 are schematic illustrations showing a medical injector having a flexible portion according to an embodiment in a first configuration and a second configuration, respectively.

FIGS. 5 and 6 are perspective views of a medical injector having a flexible portion according to an embodiment in a first configuration and a second configuration, respectively.

FIG. 7 is a cross-sectional view of a portion of the medical injector labeled as region Z in FIG. 6.

FIG. 8 is a cross-sectional view of a portion of a tubular member according to an embodiment, including a piston having a diameter less than a diameter of the tubular member.

FIG. 9 is a cross-sectional view of a portion of a tubular member according to an embodiment, including a piston having a diameter greater than a diameter of the tubular member.

FIG. 10 is a perspective view of a kit according to an embodiment.

FIG. 11 is a perspective view of a tubular member shown in FIG. 10 coupled to a control member shown in FIG. 10 and a needle assembly shown in FIG. 10.

FIG. 12 is a cross-sectional view of a portion of the tubular member and control member shown in FIG. 11 taken along line X-X in FIG. 11.

FIG. 13 is a perspective view of the needle assembly shown in FIG. 10.

FIG. 14 is a flow chart of a method of producing a tubular member according to an embodiment

FIGS. 15-17 are schematic illustrations of a tubular member produced according to the method illustrated in FIG. 14.

FIGS. 18 and 19 are schematic illustrations showing a flexible tubular assembly according to an embodiment in a first configuration and a second configuration, respectively.

FIG. 20 is a schematic illustration showing a portion of a medical injector according to an embodiment.

### DETAILED DESCRIPTION

In some embodiments, an apparatus includes a tubular member and a spherical piston disposed within a lumen defined by the tubular member. The tubular member includes a distal end portion and a central portion. The distal end portion is configured to be coupled to a needle. At least the central portion of the tubular member is curved. The spherical piston is movably disposed with the lumen such that the lumen is divided into a first portion and a second portion. The first portion of the lumen configured to contain a medicament. In use, the medicament can be injected from the first portion of the tubular member into a body via the needle when the spherical piston moves within the lumen.

In some embodiments, an apparatus includes a tubular member and a spherical piston disposed within a lumen defined by the tubular member. The tubular member includes a distal end portion and a central portion. The distal end portion is configured to be coupled to a needle. At least the central portion of the tubular member is flexible. In some embodiments, for example, the central portion is configured to be coiled about an axis substantially normal to a center line of the tubular member through at least one revolution. The spherical piston is movably disposed with the lumen such that the lumen is divided into a first portion and a second portion. The first portion of the lumen configured to contain a medicament. The piston is configured to contact an inner surface of the tubular member solely along a locus of points. In some embodiments, for example, the piston and the inner surface of the tubular member collectively form a substantially fluid-tight seal between the first portion of the lumen and the second portion of the lumen.

In some embodiments, a method includes filling a tubular member with a medicament. The medicament can be, for example, a dermal filler having a nominal viscosity of at least 1000 centipoise. The tubular member is cut into a first section and a second section, with at least the first section being filled with the medicament. The first section of the tubular member has a predetermined length. In some embodiments, for example, the predetermined length of the first section is such that a volume of the medicament within the first section is at least 1 cubic centimeter. In other embodiments, for example, the predetermined length of the first section is such that a volume of the medicament within the first section is at least 5 cubic centimeters.

In some embodiments, the method optionally includes attaching a fitting to a first end portion of the first section. The fitting is configured to removably couple the first end portion of the first section to a needle. In some embodiments, the method optionally includes disposing a piston within a lumen defined by the first section of the tubular member, such that the lumen of the first section of the tubular member is divided into a first portion and a second portion, with the first portion of the lumen containing the medicament. In some embodiments, the method optionally includes coupling a cap to the fitting such that the first portion of the lumen of the first section is fluidically isolated from a region outside of the first section of the tubular member.

As used herein, the words “proximal” and “distal” refer to direction closer to and away from, respectively, an operator (e.g., surgeon, physician, nurse, technician, etc.) of the medical device. Thus, for example, the end of the medicament delivery device contacting the patient's body would be the distal end of the medicament delivery device, while the end opposite the distal end would be the proximal end of the medicament delivery device.

As used herein, the words “flexibility” and “flexible” are used to describe a structure having a first portion that is easily deflected, displaced and/or deformed with respect to a second portion when an external load is applied to the first portion of the structure. For example, a first tubular member having a greater degree of flexibility is less resistant to deflection when exposed to a force than a second tubular member having a lesser degree of flexibility. Thus, in some embodiments, the first (or more flexible) tubular member can be more easily coiled and/or coiled to a smaller radius of curvature than the second (or less flexible) tubular member.

In some embodiments, the flexibility of an object and/or system can be characterized by the object's and/or the system's linear flexibility. The linear flexibility can be characterized in terms of the amount of force applied to a first portion the object and/or system and the resulting linear distance through which the first portion of the object and/or the system deflects, deforms, and/or displaces with respect to a second portion of the object and/or system. In other embodiments, the flexibility of an object and/or system can be characterized by the object's and/or the system's rotational (or torsional) flexibility. The rotational flexibility can be characterized in terms of the torque (or moment) applied to the object and/or the system and the resulting magnitude of rotation (i.e., the angle of rotation) of the first portion of the object and/or the system with respect to a second portion of the object and/or the system.

The flexibility of an object is an extensive property of the object, and thus is dependent upon both the material from which the object is formed and/or certain physical characteristics of the object (e.g., the shape of portions of the object). For example, the flexibility of an object can be increased by constructing the object from a material having a low modulus of elasticity or a low flexural modulus. For example, a tube constructed of urethane can be more flexible than a similar tube constructed of stainless steel, because the modulus of elasticity and/or the flexural modulus of urethane is significantly less than the modulus of elasticity and/or the flexural modulus of urethane of stainless steel. The flexibility of an object can also be increased by changing a physical characteristic of the object and/or the components from which the object is constructed. In certain instances, the flexibility of an object can be increased by changing the shape and/or size of the components from which an object is constructed. For example, a tube constructed of braided steel fibers can be more flexible than a similar tube constructed from monolithically extruded steel, because the shape and/or size of the braided steel fibers can provide greater flexibility than a monolithic construction of the same material. As another example, a tube constructed from a polymer (e.g., polyurethane) and having a wall thickness of 0.5 mm can be more flexible than a tube constructed from the same material and having a wall thickness of 2 mm.

In certain instances, a flexible object can be an object that is easily elastically deformed when an external load is applied to the object. For example, in certain instances, a tubular member constructed from an elastomeric material that can easily stretch (i.e., elastically deform), linearly and/or radially, when an external load is applied to the object. In addition to being considered a flexible tubular member, the tubular member can also be considered as “elastic” or “resilient.” In other instances, a flexible object can be an object that is easily plastically deformed when an external load is applied to the object. In yet other instances, a flexible object can be constructed from one or more components and/or materials characterized as “rigid.” Said another way, in certain instances, a flexible object can be constructed from one or more components and/or materials having a high modulus of elasticity, a high flexural modulus, and/or a high yield strength. For example, in certain instances, a tubular member can be constructed from multiple steel tubes that are coupled together such that the overall tubular member is easily deflected, displaced and/or deformed when an external load is applied to the tubular member.

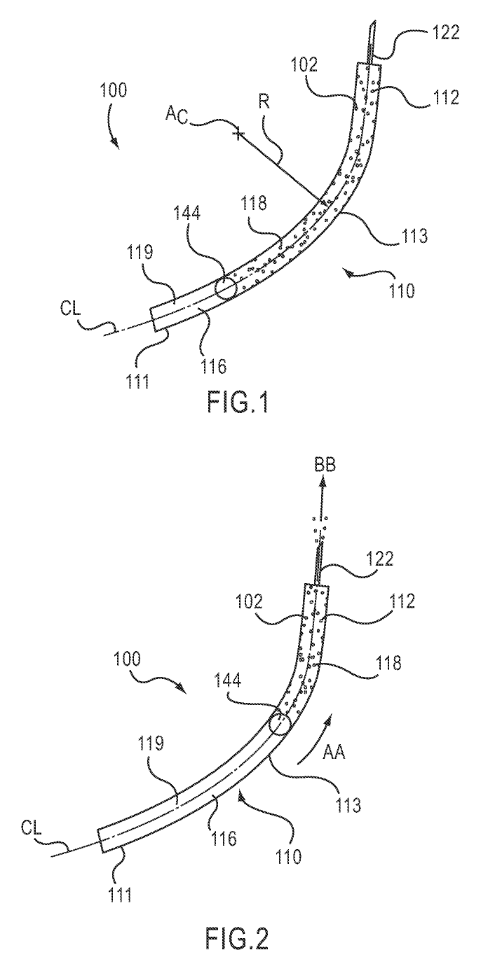

FIGS. 1 and 2 are schematic illustrations of a medical injector 100 according to an embodiment in a first configuration and a second configuration, respectively. The medical injector 100 includes a tubular member 110, a spherical piston 144 and a needle 122. The tubular member 110 defines a lumen 116 therethrough having a longitudinal center line CL. For clarity, the tubular member 110 is shown as being clear so that components therein (e.g., the piston 144) can be shown. The tubular member 110 includes a proximal end portion 111, a distal end portion 112, and a central portion 113 therebetween. As shown in FIGS. 1 and 2, at least the central portion 113 of the tubular member 110 is curved. More specifically the central portion 113 of the tubular member 110 is curved about an axis of curvature A_{C} that is substantially normal to the center line CL. Thus, at least a portion of the central portion 113 and/or the center line CL has a radius of curvature R about the axis of curvature A_{C}. Although the tubular member 110 is shown as being curved about a single axis of curvature A_{C}, in other embodiments, the central portion 113 can be curved about multiple axes. Moreover, although the tubular member 110 is shown in FIGS. 1 and 2 as a two-dimensional schematic, in some embodiments, a tubular member 110 can be curved about multiple axes that are not parallel to each other, thus resulting in a tubular member having a three-dimensional curvature.

The spherical piston 144 is disposed within the lumen 116 of the tubular member 110 such that the tubular member 110 is divided into a first portion 118 and a second portion 119. A medicament 102 is disposed in the first portion 118 of the tubular member 110. The medicament 102 can be, for example, a dermal filler, a sub-dermal filler, a therapeutic substance for mesotherapy, a sclerosant for sclerotherapy, a neurotoxin, or the like. In this manner, the first portion 118 of the tubular member 110 can function as a medicament container to contain the medicament 102. As discussed in more detail herein, in some embodiments, the spherical piston 144 can form a fluid-tight seal within the lumen 116 such that the first portion 118 of the tubular member 110 is fluidically isolated from the second portion 119 of the tubular member 110.

The needle 122 can be any suitable needle for injecting the medicament 102 into a body (not shown in FIGS. 1 and 2). For example, in some embodiments, the needle 122 can be a 27 gauge or smaller needle, and can have a length of at least 17 millimeters. The needle 122 is coupled to the distal end portion 112 of the tubular member 110 such that the needle 122 can be placed in fluid communication with the first portion 118 of the tubular member 110. The needle 122 can be coupled to the distal end portion 112 of the tubular member 110 by any suitable mechanism. For example, in some embodiments, the needle 122 can be threadedly coupled to the distal end portion 112 of the tubular member 110. In other embodiments, the needle 122 can be coupled to the distal end portion 112 of the tubular member 110 via a fitting, such as, for example, a twist-on Luer fitting (e.g., a Luer-Lok™ fitting, not shown in FIGS. 1 and 2).

The spherical piston 144 is disposed within the lumen 116 such that the spherical piston 144 can move within the lumen 116 along the center line CL. When the spherical piston 144 moves within the tubular member 110, as shown by the arrow AA in FIG. 2, the medicament 102 is conveyed from the first portion 118 of the tubular member 110, and through the needle 122, as shown by the arrow BB in FIG. 2. Said another way, a user can inject the medicament 102 into a body by actuating the medical injector 100 to cause the spherical piston 144 to move distally within the tubular member 110. In this manner, the curved tubular member 110 can function as a syringe. Because the central portion 113 of the tubular member 110 is curved, the user can position and/or orient the tubular member 110 such that the needle 122 is in the desired location and the proximal end portion 111 of the tubular member 110 does not interfere with the procedure (e.g., the proximal end portion 111 does not undesirably contact the body).

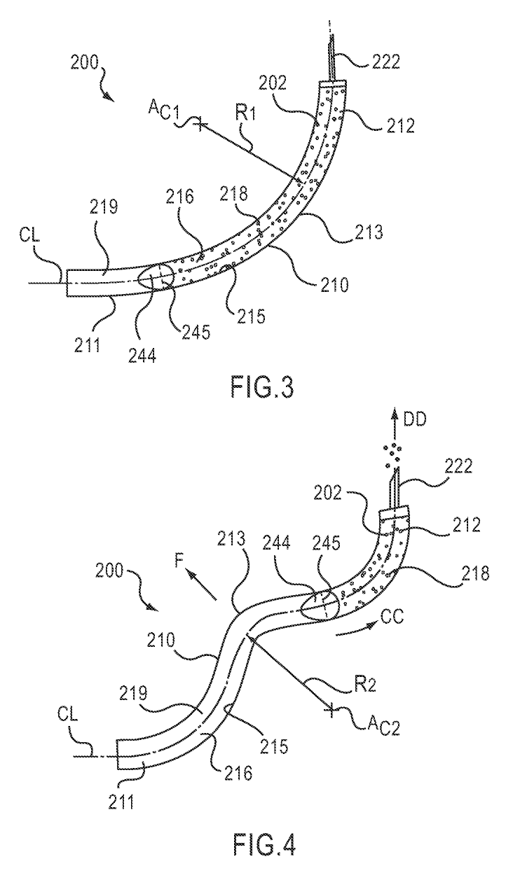

Although the tubular member 110 is shown and described above as having a substantially fixed curvature, in other embodiments, at least a portion of a tubular member can be flexible. For example, FIGS. 3 and 4 are schematic illustrations of a medical injector 200 according to an embodiment in a first configuration and a second configuration, respectively. The medical injector 200 includes a tubular member 210, a piston 244 and a needle 222. The tubular member 210 has an inner surface 215 that defines a lumen 216 having a longitudinal center line CL. The tubular member 210 includes a proximal end portion 211, a distal end portion 212, and a central portion 213 therebetween.

At least the central portion 213 of the tubular member 210 is flexible. More specifically, as shown in FIGS. 3 and 4, at least the central portion 213 of the tubular member 210 can change shape to move the tubular member 210 between a first configuration (FIG. 3) and a second configuration (FIG. 4). Similarly stated, at least the central portion 213 of the tubular member 210 can be deflected, displaced and/or deformed to move the tubular member 210 between the first configuration and the second configuration. When the tubular member 210 is in the first configuration, the central portion 213 of the tubular member 210 is curved about a first axis of curvature A_{C1} that is substantially normal to the center line CL. Thus, when the tubular member 210 is in the first configuration, at least a portion of the central portion 213 and/or the center line CL has a first radius of curvature R1 about the first axis of curvature A_{C1}.

The tubular member 210 is moved from the first configuration (FIG. 3) to the second configuration (FIG. 4) when the central portion 213 is deflected in response to an external force F. In some embodiments, the change in shape of the central portion 213 of the tubular member 210 can occur in response to relatively low force F, such as, for example, a force exerted by a user's fingers. In some embodiments, for example, the force F can be less than 5 N. Although the force F is shown as being a substantially linear force resulting in substantially linear deflection of the central portion 213 of the tubular member 210, in other embodiments, the force F can be a torsional force (i.e., a force resulting in a torque), and the central portion 213 can be rotationally flexible.

As shown in FIG. 4, when the tubular member 210 is in the second configuration, the central portion 213 of the tubular member 210 is curved about a second axis of curvature A_{C2} that is substantially normal to the center line CL. Thus, when the tubular member 210 is in the second configuration, at least a portion of the central portion 213 and/or the center line CL has a second radius of curvature R2 about the second axis of curvature A_{C2}. In this manner, the shape and/or direction of the curvature of the central portion 213 can change when the tubular member 210 is moved from the first configuration to the second configuration. Although the second axis of curvature A_{C2} is shown as being substantially parallel to the first axis of curvature A_{C1}, which results in a substantially two dimensional curvature, in other embodiments, the second axis of curvature A_{C2} can be nonparallel to the first axis of curvature A_{C1}, thus resulting in a tubular member having a three-dimensional curvature when moved from the first configuration to the second configuration. In some embodiments, the first radius of curvature R1 can be different from the second radius of curvature R2.

The needle 222 can be any suitable needle for injecting the medicament 202 into a body (not shown in FIGS. 3 and 4). The needle 222 is coupled to the distal end portion 212 of the tubular member 210 such that the needle 222 can be placed in fluid communication with the first portion 218 of the tubular member 210. The needle 222 can be coupled to the distal end portion 212 of the tubular member 210 by any suitable mechanism, as described above.

The piston 244 is disposed within the lumen 216 of the tubular member 210 such that the tubular member 210 is divided into a first portion 218 and a second portion 219. A medicament 202 is disposed in the first portion 218 of the tubular member 210. The medicament 202 can be, for example, a dermal filler, a sub-dermal filler, a therapeutic substance for mesotherapy, a sclerosant for sclerotherapy, a neurotoxin, or the like. In this manner, the first portion 218 of the tubular member 210 can function as a medicament container to contain the medicament 202.

The piston 244 includes a sealing portion 245 (shown as a dashed line in FIGS. 3 and 4) that contacts the inner surface 215 of the tubular member 210 when the piston 244 is disposed within the lumen 216. More particularly, the sealing portion 245 of the piston 244 contacts the inner surface 215 of the tubular member 210 solely along a locus of points. Similarly stated, the portion of the piston 244 that contacts the inner surface 215 of the tubular member (i.e., the sealing portion 245) approximates, within a reasonable manufacturing tolerance, a single dimension. Said another way, the sealing portion 245 of the piston 244 approximates a length dimension, and does not have a significant area (i.e., a length dimension and a width dimension). Similarly stated, the sealing portion 245 of the piston 244 has a width dimension that approximates zero within a reasonable manufacturing tolerance. By having an approximately linear sealing portion 245, the piston 244 can move within the lumen 216 through tight bends. Similarly stated, as the width dimension of the sealing portion 245 increases, the size of the radius of curvature R1, R2 through which the piston 245 can travel increases. Because the sealing portion 245 of the piston 244 is approximately linear, the piston 244 can move within the lumen 216 along a curvilinear path having a radius of curvature approximately equal to the diameter of the lumen 216. Similarly stated, this arrangement allows the piston 244 to move within the lumen 216 along the center line CL through the curved portion having a radius of curvature (e.g., R1 and/or R2) approximately equal to the diameter of the lumen 216.

In some embodiments, the sealing portion 245 of the piston 244 can extend circumferentially about the surface of the piston 244. Similarly stated, in some embodiments, the sealing portion 245 can be continuous about the surface of the piston 244. As discussed in more detail herein, in some embodiments, the sealing portion 245 and the inner surface 215 of the tubular member 210 can form a fluid-tight seal within the lumen 216 such that the first portion 218 of the tubular member 210 is fluidically isolated from the second portion 219 of the tubular member 210.

As shown by the arrow CC in FIG. 4, the piston 244 can move within the lumen 216 along the center line CL. When the piston 244 moves within the tubular member 210 the medicament 202 is conveyed from the first portion 218 of the tubular member 210, and through the needle 222, as shown by the arrow DD in FIG. 4. Said another way, a user can inject the medicament 202 into a body by actuating the medical injector 200 to cause the 244 to move distally within the tubular member 210. In this manner, the flexible tubular member 210 can function as a syringe. Because the central portion 213 of the tubular member 210 is flexible and/or curved, the user can position and/or orient the tubular member 210 such that the needle 222 is in the desired location and the proximal end portion 211 of the tubular member 210 does not interfere with the procedure (e.g., the proximal end portion 211 does not undesirably contact the body). Moreover, because the central portion 213 of the tubular member 210 is flexible, the user can change the shape of tubular member 210 when the piston 244 is moving within the lumen 210 (i.e., during an injection event).

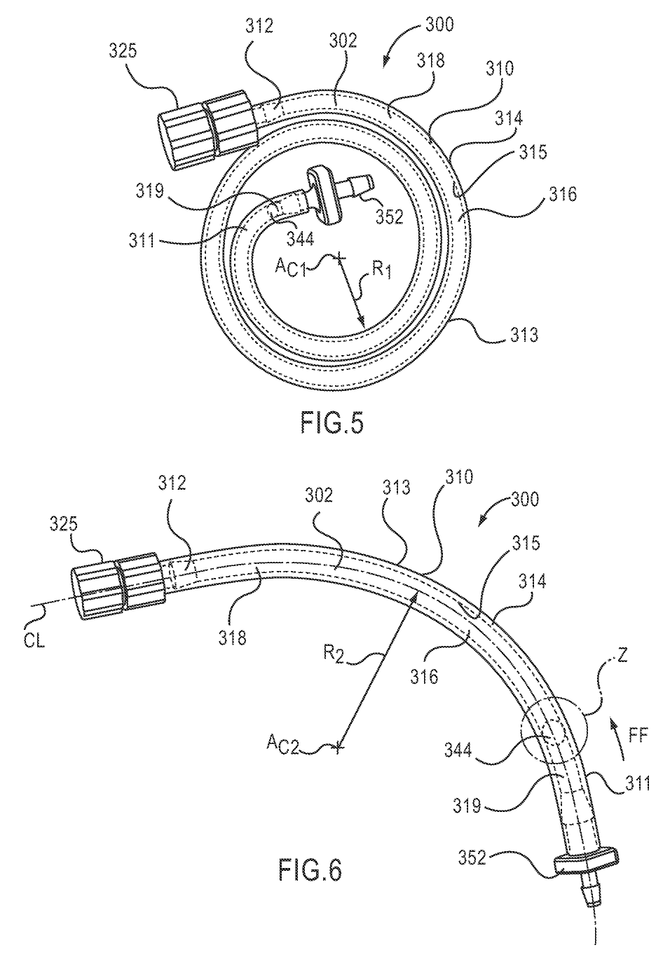

FIGS. 5 and 6 are perspective views of a medical injector 300 according to an embodiment in a first configuration and a second configuration, respectively. The medical injector 300 includes a tubular member 310, a piston 344, a distal end coupler 325, and a proximal end coupler 352. The tubular member 310 has a side wall 314 having an inner surface 315 that defines a lumen 316. The tubular member 310 defines a longitudinal center line CL and includes a proximal end portion 311, a distal end portion 312, and a central portion 313 therebetween.

At least the central portion 313 of the tubular member 310 is flexible. More specifically, as shown in FIGS. 3 and 4, at least the central portion 313 of the tubular member 310 can change shape to move the tubular member 310 between a first (coiled) configuration (FIG. 5) and a second (uncoiled) configuration (FIG. 6). Similarly stated, at least the central portion 313 of the tubular member 310 can be deflected, displaced and/or deformed to move the tubular member 310 between the first configuration and the second configuration. When the tubular member 310 is in the first configuration, the central portion 313 of the tubular member 310 is curved about a first axis of curvature A_{C1} that is substantially normal to the center line CL. Similarly stated, when the tubular member 310 is in the first configuration, the central portion 313 of the tubular member 310 is coiled such that the central portion 313 and/or the center line CL has a minimum radius of curvature R1 about the first axis of curvature A_{C1}. Similarly stated, when the tubular member 310 is in the first configuration, the central portion 313 of the tubular member 310 is coiled such that the first coil of central portion 313 has a first radius of curvature R1 about the first axis of curvature A_{C1}, while the second coil of the central portion 313 has a radius of curvature greater than R1. In this manner, the tubular member 310 can be easily stored, transported and/or handled when in the first configuration.

Although the tubular member 310 is shown as being coiled approximately two revolutions (i.e., 720 degrees) when in the first configuration, in other embodiments, the tubular member 310 can be coiled through any suitable number of revolutions. For example, in some embodiments, the tubular member 310 can be coiled through at least one revolution. In other embodiments, the tubular member 310 can be coiled between two and eight revolutions. In yet other embodiments, the tubular member 310 can be coiled between four and seven revolutions.

The number revolutions through which the tubular member 310 can be coiled can be dependent on, among other things, the length of the tubular member 310, radius of curvature R1 of the central portion 313 and/or the flexibility of the tubular member 310. In some embodiments, the tubular member 310 can be coiled such that the radius of curvature R1 is less than approximately 10 centimeters. In other embodiments, the tubular member 310 can be coiled such that the radius of curvature R1 is less than approximately 50 centimeters.

The central portion 313 of the tubular member 310 can be deflected, displaced and/or deformed to move the tubular member 310 from the first configuration (FIG. 5) to the second configuration (FIG. 6). Similarly stated, the tubular member 310 can be uncoiled when moved from the first configuration to the second configuration. In some embodiments, the central portion 313 of the tubular member 310 can be deflected, displaced and/or deformed in response to an external force (not shown in FIGS. 5 and 6). In other embodiments, the central portion 313 of the tubular member 310 can be deflected, displaced and/or deformed in response to the absence of a restraining force (i.e., a force that maintains the tubular member 310 in the first configuration).

As shown in FIG. 6, when the tubular member 310 is in the second configuration, the central portion 313 of the tubular member 310 is curved about a second axis of curvature A_{C2} that is substantially normal to the center line CL. Thus, when the tubular member 310 is in the second configuration, at least a portion of the central portion 313 and/or the center line CL has a second radius of curvature R2 about the second axis of curvature A_{C2}. Although the second axis of curvature A_{C2} is shown as being substantially parallel to the first axis of curvature A_{C1}, which results in a substantially two dimensional curvature, in other embodiments, the second axis of curvature A_{C2} can be nonparallel to the first axis of curvature A_{C1}. In some embodiments, the first radius of curvature R1 can be different from the second radius of curvature R2.

Although the second configuration of the tubular member 310 is shown in FIG. 6 has having a particular shape, the tubular member 310 can have any suitable shape when in the second configuration. When the tubular member 310 is in the second configuration, the shape of the tubular member 310 can be defined by the user, by the weight of the tubular member 310, by a predefined nominal shape of the tubular member 310 and/or any combination thereof. In this manner, the tubular member 310 can be manipulated to enhance the performance of the procedure in which the tubular member 310 is being used.

The distal end coupler 325 is coupled to the distal end portion 312 of the tubular member 310, and is configured to couple a needle (not shown in FIGS. 5 and 6) to the distal end portion 312 of the tubular member 310. The distal end coupler 325 can be any suitable coupler for coupling a needle to the tubular member 310 and/or maintaining a substantially fluid-tight seal between the needle and a first portion 318 of the tubular member 310. For example, in some embodiments, the distal end coupler 325 can removably couple a needle to the distal end portion 312 of the tubular member 310. For example, in some embodiments, the distal end coupler 325 can be a press-fit Luer fitting (e.g., a Luer-Slip™ fitting), a twist-on Luer fitting (e.g., a Luer-Lok™ fitting), a barbed Luer adapter and/or the like.

The proximal end coupler 352 is coupled to the proximal end portion 311 of the tubular member 310, and is configured to operatively couple an energy source (not shown in FIGS. 5 and 6) to the proximal end portion 311 of the tubular member 310. The energy source can be any suitable source that produces a kinetic energy to move the piston 344 within the lumen 316 of the tubular member 310, as described in more detail herein. For example, in some embodiments, the energy source can include a pressurized gas that exerts a force on the piston 344, thereby causing the piston 344 to move within the lumen 316. In such embodiments, the proximal end coupler 352 couples the source of pressurized gas to the proximal end portion 311 of the tubular member 310 such that a second portion 319 of the lumen 316 can be placed in fluid communication with the source of pressurized gas. Moreover, in such embodiments, the proximal end coupler 352 can form a substantially fluid-tight seal between the second portion 319 of the lumen 316 and the source of pressurized gas. In other embodiments, the proximal end coupler 352 and the energy source can be any coupler and energy source, respectively, of the types shown and described in U.S. patent application Ser. No. 12/114,194, entitled “Apparatus and Methods for Injecting High Viscosity Dermal Fillers,” filed May 2, 2008, which is incorporated herein by reference in its entirety.

The piston 344 is disposed within the lumen 316 of the tubular member 310 such that the tubular member 310 is divided into the first portion 318 and the second portion 319. A medicament 302 is disposed in the first portion 318 of the tubular member 310. The medicament 302 can be, for example, a dermal filler, a sub-dermal filler, a therapeutic substance for mesotherapy, a sclerosant for sclerotherapy, a neurotoxin, or the like. In this manner, the first portion 318 of the tubular member 310 can function as a medicament container to contain the medicament 302.

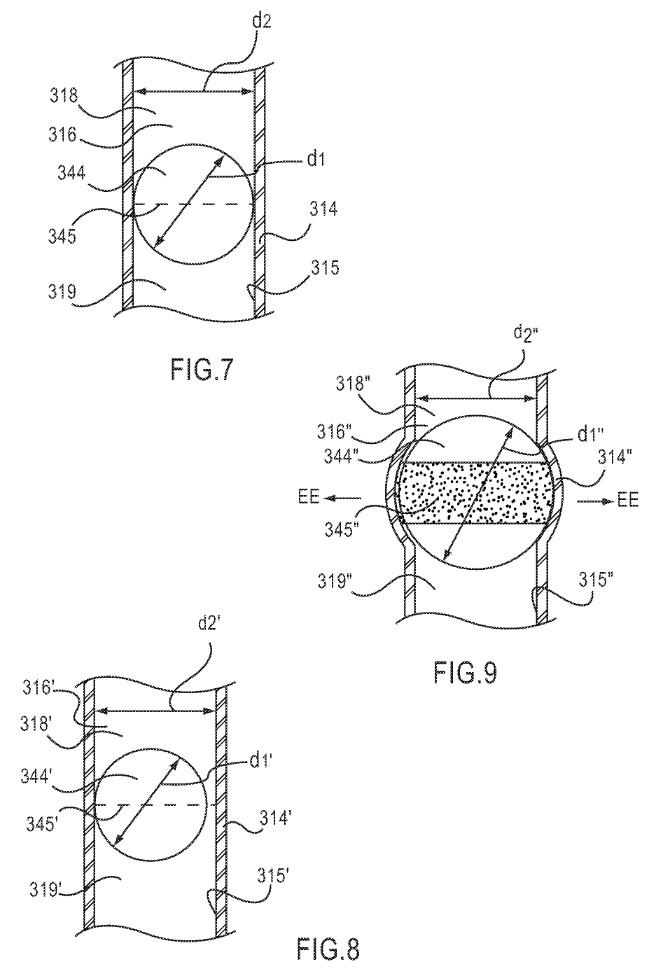

As shown in FIG. 7, the piston 344 is substantially spherical and has a diameter d1 that is substantially equal to the diameter d2 of the lumen 316. Thus, when the piston 344 is disposed within the lumen 316, a portion of the surface of the piston 344 contacts the inner surface 315 of the tubular member. More particularly, as described above, the piston 344 includes a sealing portion 345 that substantially circumscribes the outer surface of the piston 344 and contacts the inner surface 315 of the tubular member 310. As described above, the sealing portion 345 contacts the inner surface 315 along an approximate locus of points. Similarly stated, the sealing portion 345 of the piston 344 that contacts the inner surface 315 of the tubular member 310 approximates, within a reasonable manufacturing tolerance, a single dimension. Said another way, the sealing portion 345 of the piston 344 does not have a significant area (i.e., a length dimension and a width dimension). Similarly stated, the sealing portion 345 of the piston 344 has a width dimension that approximates zero within a reasonable manufacturing tolerance. In this manner, the sealing portion 345 and the inner surface 315 of the tubular member 310 can form a fluid-tight seal within the lumen 316 such that the first portion 318 of the tubular member 310 is fluidically isolated from the second portion 319 of the tubular member 310.

Although the diameter d1 is shown and described as being substantially equal to the diameter d2 of the lumen 316, in other embodiments, the diameter d1 can be different than the diameter d2 of the lumen 316. For example, as shown in FIG. 8, in some embodiments, the diameter d1′ of the piston 344′ can be smaller than the diameter d2′ of the lumen 316′. In such embodiments, the portion of the piston 344′ contacting the inner surface of the lumen does not circumscribe the outer surface of the piston 344′. Said another way, in such embodiments, the sealing portion 345′ of the piston 344′ and the inner surface 315′ of the tubular member 310′ do not form a fluid-tight seal within the lumen 316′. In some such embodiments, the viscosity of the medicament 302 can be such that even in the absence of a fluid-tight seal, the medicament does not readily flow from the first portion 318 of the tubular member 310 into the second portion 319 of the tubular member 310 during use.

As shown in FIG. 9, in other embodiments, the diameter d1″ of the piston 344″ can be greater than the diameter d2″ of the lumen 316″. In such embodiments, the sealing portion 345″ (shown as the shaded region in FIG. 9) of the piston 344″ is a two-dimensional region. Said another way, the sealing portion 345″ of the piston 344″ has a length dimension and a width dimension. Thus, the sealing portion 345″ of the piston 344″ contacts the inner surface 315″ of the tubular member 310″ along a two-dimensional area. Moreover, as shown by the arrows EE in FIG. 9, to accommodate the placement of the piston 344″ within the lumen 316″, the tubular member 310″ elastically and/or plastically deforms in the region where the inner surface 315″ contacts the sealing portion 345″. Thus, the tubular member 310″ is both flexible (i.e., can readily changes shapes between the first configuration and the second configuration) and deformable. In this manner, the sealing portion 345″ and the inner surface 315″ of the tubular member 310″ can form a fluid-tight seal within the lumen 316″. Moreover, such an arrangement can allow the integrity of the fluid-tight seal to be maintained at high injection pressures (e.g., a pressure within the first portion 318″ of the lumen 316″ greater than 70 p.s.i.). In some embodiments, for example, the diameter d1″ can be approximately 0.13 mm (0.005 inches) greater than the diameter d2″.

As shown by the arrow FF in FIG. 6, the piston 344 can move within the lumen 316 along the center line CL. When the spherical piston 344 moves within the tubular member 310 the medicament 302 can conveyed from the first portion 318 of the tubular member 310, as described above. Said another way, a user can inject the medicament 302 into a body by actuating the medical injector 300 to cause the piston 344 to move distally within the tubular member 310. In this manner, the flexible tubular member 310 can function as a syringe. Moreover, because the central portion 313 of the tubular member 310 is flexible, the user can change the shape of tubular member 310 when the piston 344 is moving within the lumen 310 (i.e., during an injection event).

The thickness of the side wall 314 of the tubular member 310 can have any suitable value to withstand a desired injection pressure, and/or to maintain a desired level of flexibility. In some embodiments, for example, the thickness of the side wall 314 can be such that the tubular member 314 can withstand an injection pressure (i.e., the pressure of the medicament 302 during an injection event) of up to 689 kPa (100 p.s.i.), 1378 kPa (200 p.s.i.), 1722 kPa (250 p.s.i.), 3445 kPa (500 p.s.i.), 6890 kPa (1000 p.s.i.), 13.8 MPa (2000 p.s.i.) and/or 34.5 MPa (5000 p.s.i.). In some embodiments, for example, the thickness of the side wall 314 can be between 1 mm and 3 mm. In other embodiments, the thickness of the side wall 314 can be between 1 mm and 1.5 mm. In yet other embodiments, the thickness of the side wall 314 can be between 1.5 mm and 2.5 mm.

The diameter d2 of the lumen 316 and the length of the tubular member 310 can be selected such that the tubular member 310 can contain the desired volume of the medicament 302. For example, in some embodiment, the diameter d2 of the lumen 316 and the length of the tubular member 310 can be selected such that the first portion 318 of the tubular member 310 can contain approximately 0.5 cubic centimeters, 1 cubic centimeter, 2 cubic centimeters, 3 cubic centimeters, 5 cubic centimeters, 10 cubic centimeters, 15 cubic centimeters and/or 20 cubic centimeters of the medicament 302. In some embodiments, the tubular member 310 can contain greater than 20 cubic centimeters of the medicament. In some embodiments, for example, the diameter d2 of the lumen can be between approximately 0.5 mm and approximately 10 mm. In other embodiments, the diameter d2 of the lumen can be between approximately 1 mm and approximately 8 mm. In yet other embodiments, the diameter d2 of the lumen can be between approximately 1 mm and approximately 6 mm. In yet other embodiments, the diameter d2 of the lumen can be between approximately 2 mm and approximately 6 mm. In yet other embodiments, the diameter d2 of the lumen can be between approximately 0.8 mm and approximately 2 mm. In yet other embodiments, the diameter d2 of the lumen can be between approximately 1 mm and approximately 2 mm. In some embodiments, the length of the tubular member 310 can be approximately 10 centimeters. In other embodiments, the length of the tubular member 310 can be at least approximately 50 centimeters. In yet other embodiments, the length of the tubular member 310 can be at least approximately 1 m. In yet other embodiments, the length of the tubular member 310 can be at least approximately 2 m. In yet other embodiments, the length of the tubular member 310 can be at least approximately 3 m.

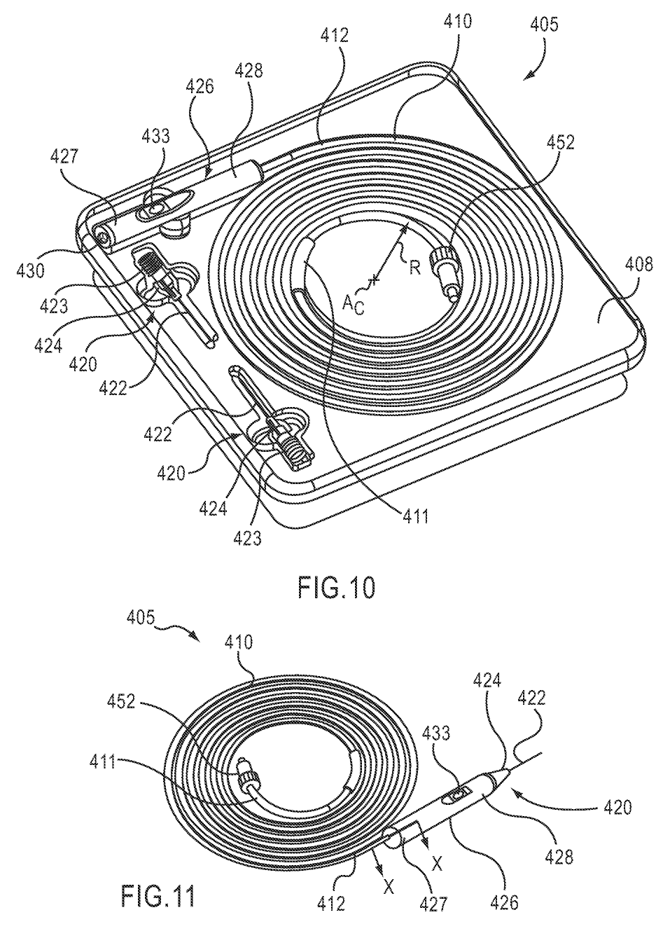

FIGS. 10-13 show a kit 405 according to an embodiment. The kit 405 includes a tubular member 410, a control member 426, a proximal end coupler 452 and two needle assemblies 420, each of which can be disposed within a container 408. As described in more detail below, the tubular member 410 can be pre-filled with a medicament, such as, for example a dermal filler. In some embodiments, the components of the kit 405 can be sealed within the container 408 such that the components (e.g., the needle assemblies 420) remain sterile until the container 408 is opened.

The tubular member 410 is flexible and thus can change shape to fit within the container 408, to accommodate movement of the control member 426 during a medical procedure, or the like. Similarly stated, at least a portion of the tubular member 410 can be deflected, displaced and/or deformed to move the tubular member 410 between any number of different configurations. As shown in FIG. 10, the tubular member 410 can be coiled about an axis of curvature A_{C}. Similarly stated, when the tubular member 410 is disposed within the container 408, the tubular member 410 is coiled such that the tubular member 410 has a minimum radius of curvature R about the axis of curvature A_{C}.

When the tubular member 410 is removed from the container 408 and/or when the tubular member 410 is in use, at least a portion of the tubular member 410 can be deflected, displaced and/or deformed to move the tubular member 410 from the coiled shape into any suitable shape. Said another way, the tubular member 410 can be uncoiled when removed from the container 408. In some embodiments, the portion of the tubular member 410 can be deflected, displaced and/or deformed in response to an external force. Similarly stated, in some embodiments, the coiled shape can be the nominal (or default) shape of the tubular member 410, and the tubular member 410 can change shape when an external force is applied thereto. In other embodiments, the portion of the tubular member 410 can be deflected, displaced and/or deformed in response to the absence of an external force. Similarly stated, in such embodiments, the tubular member 410 does not have a nominal (or default) shape, or the nominal (or default shape) is different than the coiled shape.

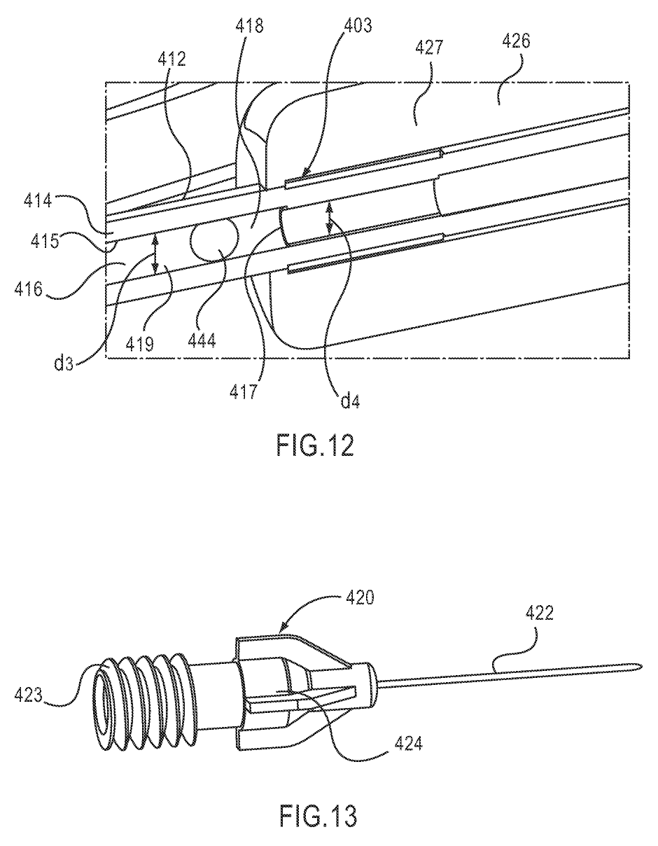

As shown in FIG. 12, which is a cross-sectional view of a portion of the tubular member 410 and the control member 426 taken along line X-X in FIG. 11, the tubular member 410 has a side wall 414 having an inner surface 415 that defines a lumen 416. A piston 444 is disposed within the lumen 416 of the tubular member 410 such that the tubular member 410 is divided into the first portion 418 and the second portion 419 (see e.g., FIG. 12). A medicament (not shown in FIGS. 10-13) is disposed in the first portion 418 of the tubular member 410. Similarly stated, the first portion 418 of the tubular member 410 can be pre-filled with the medicament, such as, for example, a dermal filler.

A sleeve 403 is disposed about a distal end portion 412 of the tubular member 410. The sleeve 403 can be any suitable rigid member, such as, for example, a thin-walled steel tube. As shown in FIG. 12, the portion of the tubular member 410 about which the sleeve 403 is disposed has a lumen diameter d4 that is less than the nominal diameter d3 of the lumen 416. Additionally, the transition between the nominal diameter d3 and the reduced lumen diameter d4 is not gradual, but occurs at a shoulder 417. This arrangement limits the movement of the piston 444 within the lumen 416. More particularly, the distal movement of the piston 444 will be limited when the piston 444 contacts the shoulder 417. In some embodiments, sleeve 403 can be disposed about the tubular member 410 at a predetermined longitudinal position to limit the volume of medicament that can be injected during a procedure. For example, when the sleeve 403 is positioned at a more proximal location along the tubular member 410, the distance through which the piston 444 can travel within the lumen 416 is reduced, thereby reducing the amount of medicament that can be injected from the tubular member 410.

Returning to FIGS. 10 and 11, the proximal end coupler 452 is coupled to a proximal end portion 411 of the tubular member 410, and is configured to operatively couple an energy source (not shown in FIGS. 10-13) to the proximal end portion 411 of the tubular member 410. The energy source can be any suitable source that produces a kinetic energy to move the piston 444 within the lumen 416 of the tubular member 410 such that the medicament can be conveyed from the tubular member 410. For example, as described above, in some embodiments, the energy source can include a pressurized gas that exerts a force on the piston 444, thereby causing the piston 444 to move within the lumen 416.

The control member 426 is coupled to a distal end portion 412 of the tubular member 410. The control member 426 is configured to couple a needle assembly 420 to the distal end portion 412 of the tubular member 410 and to control the flow of medicament through the needle assembly 420 during a procedure. The control member 426 includes a first end portion 427 and a second end portion 428. The first end portion 427 includes a threaded fitting 430 configured to threadedly engage a threaded portion 423 of the needle hub 424 (see e.g., FIG. 13). In this manner, the needle assembly 420 can be removably coupled to the control member 426. The control member 426 defines a flow passageway (not shown) such that when the needle assembly 420 is coupled to the control member 426, the needle assembly 420 can be placed in fluid communication with the first portion 418 of the tubular member 410.

The second end portion 428 of the control member 426 is coupled to the distal end portion 412 of the tubular member 410. In some embodiments, the control member 426 can be fixedly coupled to the tubular member 410. In other embodiments, the control member 416 can be removably coupled to the tubular member 410.

The outer surface of the control member 426 is configured to be grasped and/or manipulated by a user. The outer surface of the control member 426 includes a switch 433 to allow the user to control the flow and/or pressure of the medicament through the needle assembly 420 during an injection event. In this manner, the user can adjust the amount the medicament being injected within and/or beneath the skin to provide the desired cosmetic and/or therapeutic results. The control member 426 can include any suitable control mechanism for controlling the flow of medicament. For example, in some embodiments, the control member 426 can control the transmission of energy from the energy source to the piston 444. In other embodiments, the control member 426 can selectively restrict the flow path of the medicament through the control member 426 and/or the needle assembly 420. The control member 426 can include any flow and/or pressure control mechanism of the types shown and described in U.S. patent application Ser. No. 12/114,194, entitled “Apparatus and Methods for Injecting High Viscosity Dermal Fillers,” filed May 2, 2008, which is incorporated herein by reference in its entirety. In yet other embodiments, the control member 426 can control the flow and/or pressure of the medicament through the control member 426 and/or the needle assembly 420 by regulating the temperature of the medicament. For example, in some embodiments, the control member 426 can include a heater (not shown) to selectively control the temperature of the medicament as it flows through the control member 426 and/or the needle assembly 420. The heater can be any heater of the types shown and described in International Patent Application No. PCT/US2007/023226, entitled “Compositions, Devices and Methods for Modifying Soft Tissue,” filed Nov. 1, 2007, which is incorporated herein by reference in its entirety. In yet other embodiments, the control member 426 can control the flow and/or pressure of the medicament through the control member 426 and/or the needle assembly 420 by applying a controlled vibration (e.g., an oscillating force) to the medicament. In this manner, the viscosity of certain medicaments (e.g., certain non-Newtonian dermal fillers) can be decreased to allow a higher flow rate of the medicament through the control member 426 and/or the needle assembly 420.

As shown in FIG. 13, the needle assembly 420 includes a needle hub 424, and a needle 422. The needle hub 424 includes a threaded portion 423 configured to be threadedly coupled to the threaded fitting 430 of the control member 426, as described above. The needle 422 can be any suitable needle for injecting the medicament into a body. For example, in some embodiments, the needle 422 can be a 27 gauge or smaller needle, and can have a length of at least 17 millimeters. In some embodiments, the two needle assemblies 420 included in the kit 405 can have the same sizes and characteristics. In other embodiments, the kit 405 can contain two or more needles having different sizes and/or characteristics. In this manner, a user can select a needle assembly 420 that is most appropriate for the procedure being performed.

As discussed above, the diameter d3 of the lumen 416 and the length of the tubular member 410 can be selected such that the tubular member 410 can contain the desired volume of the medicament. For example, in some embodiment, the diameter d3 of the lumen 416 and the length of the tubular member 410 can be selected such that the first portion 418 of the tubular member 410 can contain approximately 0.5 cubic centimeters, 1 cubic centimeter, 2 cubic centimeters, 3 cubic centimeters, 5 cubic centimeters, 10 cubic centimeters, 15 cubic centimeters and/or 20 cubic centimeters of the medicament 402. In some embodiments, the tubular member 410 can contain greater than 20 cubic centimeters of the medicament 402. In some embodiments, for example, the diameter d3 of the lumen 416 can be between approximately 0.5 mm and approximately 10 mm. In other embodiments, the diameter d3 of the lumen 416 can be between approximately 1 mm and approximately 8 mm. In yet other embodiments, the diameter d3 of the lumen 416 can be between approximately 1 mm and approximately 6 mm. In yet other embodiments, the diameter d3 of the lumen 416 can be between approximately 2 mm and approximately 6 mm. In yet other embodiments, the diameter d3 of the lumen 416 can be between approximately 0.8 mm and approximately 2 mm. In yet other embodiments, the diameter d3 of the lumen 416 can be between approximately 1 mm and approximately 2 mm. In some embodiments, the length of the tubular member 410 can be approximately 10 cm. In other embodiments, the length of the tubular member 410 can be at least approximately 50 cm. In yet other embodiments, the length of the tubular member 410 can be at least approximately 1 m. In yet other embodiments, the length of the tubular member 410 can be at least approximately 2 m. In yet other embodiments, the length of the tubular member 410 can be at least approximately 3 m.

Although the kit 405 is shown and described as including one tubular member 410, in other embodiments, a kit can include any number of tubular members pre-filled with a medicament. For example, in some embodiments, a kit can include a first tubular member containing approximately 2 cubic centimeters of a dermal filler, a second tubular member containing approximately 4 cubic centimeters of a dermal filler, and a third tubular member containing approximately 10 cubic centimeters of a dermal filler. In this arrangement, a user can select the tubular member having the desired volume of dermal filler.

Moreover, in some embodiments, a kit can include multiple pre-filled tubular members that can be coupled together to form a single tubular assembly having a first end that can be coupled to a needle and a second end that can be coupled to an actuator. Similarly stated, in some embodiments, a kit can include multiple pre-filled tubular members that can form a modular tubular assembly. In this manner, a user can assemble a medicament container containing the desired volume of medicament. For example, in a procedure that requires approximately 4 cubic centimeters of a dermal filler to be injected, a user can couple two pre-filled tubular members together, each containing approximately 2 cubic centimeters, to form a medicament container and/or a medicament injector containing the desired volume of dermal filler.

In other embodiments, a kit can include multiple pre-filled tubular members containing different medicaments that can be coupled together to form a single tubular assembly having a first end that can be coupled to a needle and a second end that can be coupled to an actuator. Similarly stated, in some embodiments, a kit can include multiple pre-filled tubular members containing different medicaments that can form a modular tubular assembly. In this manner, a user can assemble a medicament container containing the desired volume and/or types of medicament. For example, in a procedure that requires approximately 2 cubic centimeters of a first dermal filler and 3 cubic centimeters of a second dermal filler (different than the first dermal filler) to be injected, a user can couple two pre-filled tubular members together, the first pre-filled tubular member containing 2 cubic centimeters of the first dermal filler and the second pre-filled tubular member containing 3 cubic centimeters of the second dermal filler, to form a medicament container and/or a medicament injector containing the desired volume and types of dermal fillers.

In yet other embodiments, a kit can include pre-filled tubular members containing other therapeutic agent, such as for example, lidocaine. In such embodiments, for example, a user can assemble a medicament container containing the desired volume and/or types of medicament and a therapeutic agent. The therapeutic agent can be included, for example, as the distal-most tubular member. In this manner, when the injection begins the therapeutic agent can be injected before the dermal filler.

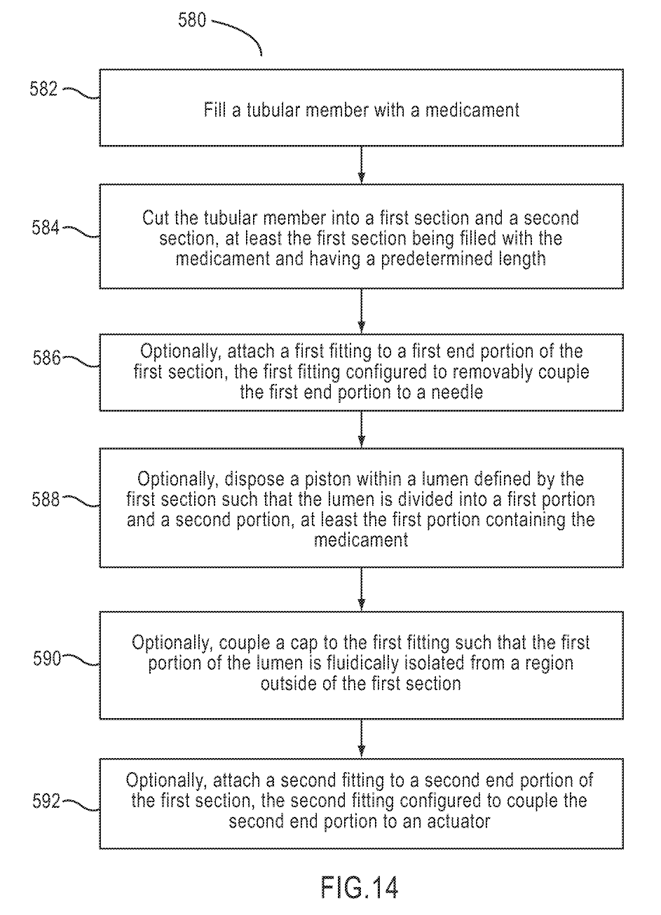

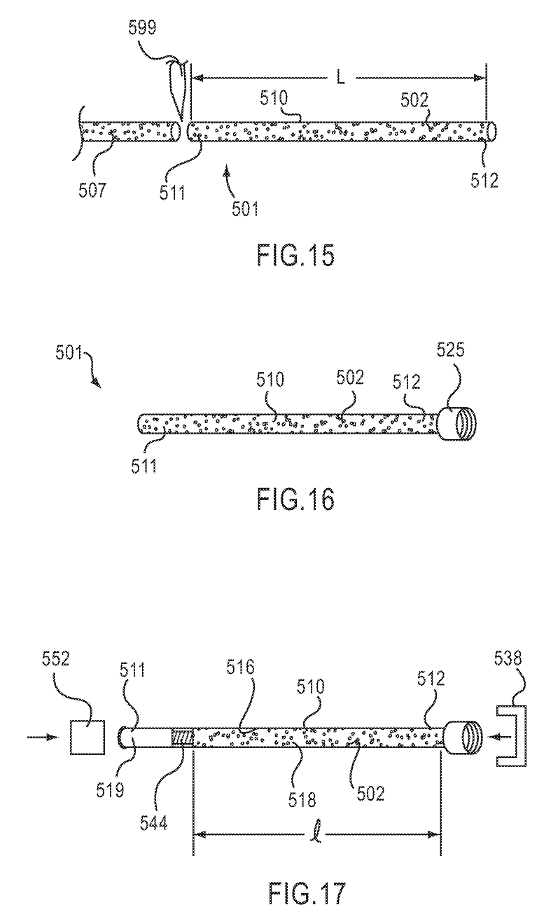

FIG. 14 is a flow chart illustrating a method 580 of producing a tubular member filled with a medicament according to an embodiment. FIGS. 15-17 are schematic illustrations showing a tubular member 510 containing a medicament and having a predetermined length L produced according to the method 580. As shown in FIG. 14, the illustrated method includes filling a tubular member with a medicament, at 582. The tubular member can be any tubular member of the types shown and described herein. For example, in some embodiments, the tubular member can be a linear tubular member that is substantially inflexible. In other embodiments, the tubular member can have a curved portion. In yet other embodiments, the tubular member can have a flexible portion. The medicament can be, for example, a dermal filler, a sub-dermal filler, a therapeutic substance for mesotherapy, a sclerosant for sclerotherapy, a neurotoxin, or the like. In some embodiments, the medicament can be a dermal filler having a nominal viscosity (i.e., the viscosity of the dermal filler at the conditions in which the tubular member is filled with the medicament) of at least 1000 centipoise.

The tubular member can be filled with the medicament using any suitable method. For example, in some embodiments, the tubular member can be a flexible tubular member stored on a roll, spool, or the like. In such embodiments, a first end portion of the tubular member can be fluidically coupled to a source of the medicament. The medicament can be conveyed from the source of the medicament into the tubular member while the tubular member remains wrapped on the roll, spool, or the like. In other embodiments, the tubular member can be wrapped about a roll, spool or the like during and/or after the tubular member is filled with the medicament. In this manner, a long tubular member can be filled in a continuous manner without taking up a large amount of space. In some embodiments, for example, the tubular member can be at least 10 meters, at least 50 meters, at least 100 meters, at least 200 meters and/or at least 500 meters.

The tubular member is cut into a first section and a second section, at 584. Referring to FIG. 15, the tubular member 501 is cut into a first section 510 and a second section 507 by a cutter 599. At least the first section 510 of the tubular member 501 is filled with a medicament 502. The first section 510 of the tubular member is cut to have a predetermined length L. The length L of the first section 510 of the tubular member 501 can be selected such that the first section 510 contains the desired volume of the medicament. In this manner, a medicament container filled with a predetermined volume of medicament can be produced without the need for filling equipment that controls the flow rate and/or fill time of medicament into the medicament container. Similarly stated, in this manner, a medicament container filled with a predetermined volume of medicament can be produced by a continuous filling operation (e.g., a fill operation that does not include multiple valves to control the flow rate of the medicament into the medicament container). For example, in some embodiments, the length L of the first section 510 of the tubular member 501 can be selected such that the first section 510 of the tubular member 501 contains approximately 0.5 cubic centimeters, 1 cubic centimeter, 2 cubic centimeters, 3 cubic centimeters, 5 cubic centimeters, 10 cubic centimeters, 15 cubic centimeters and/or 20 cubic centimeters of the medicament 502. In some embodiments, the first section 510 can contain greater than 20 cubic centimeters of the medicament 502.

In some embodiments, the length L of the first section 510 tubular member 501 can be approximately 10 cm. In other embodiments, the length L of the first section 510 can be at least approximately 50 cm. In yet other embodiments, the length L of the first section 510 can be at least approximately 1 m. In yet other embodiments, the length L of the first section 510 can be at least approximately 2 m. In yet other embodiments, the length L of the first section 510 can be at least approximately 5 m.

The volume of the medicament contained with the first section 510 of the tubular member 501 is also a function of the diameter of the lumen of the first section 510. In some embodiments, for example, the diameter of the lumen can be between approximately 0.5 mm and approximately 10 mm. In other embodiments, the diameter of the lumen can be between approximately 1 mm and approximately 8 mm. In yet other embodiments, the diameter of the lumen can be between approximately 1 mm and approximately 6 mm. In yet other embodiments, the diameter of the lumen can be between approximately 2 mm and approximately 6 mm. In yet other embodiments, the diameter of the lumen can be between approximately 0.8 mm and approximately 2 mm. In yet other embodiments, the diameter of the lumen can be between approximately 1 mm and approximately 2 mm.

Returning to the flow chart of FIG. 14, in some embodiments, the method optionally includes attaching a first fitting to a first end portion of the first section of the tubular member, at 586. As described above, the first fitting is configured to removably couple the first end portion of the first section to a needle. Referring to FIG. 16, the first fitting 525 is coupled to the first end portion 512 of the first section 510 of the tubular member 501. The first fitting 525 can be, for example, a press-fit Luer fitting (e.g., a Luer-Slip™ fitting), a twist-on Luer fitting (e.g., a Luer-Lok™ fitting), a barbed Luer adapter and/or the like.

Returning to the flow chart of FIG. 14, in some embodiments, the method optionally includes disposing a piston within a lumen defined by the first section of the tubular member, at 588. In this manner, the lumen is divided into a first portion and a second portion. At least the first portion of the lumen contains the medicament. Referring to FIG. 17, the piston 544 is disposed within the lumen 516 of the first section 510 of the tubular member 501. In some embodiments, the piston 544 can be, for example, a spherical piston of the types shown and described above. In other embodiments, the piston 544 can be a cylindrical piston, and/or can have sealing rings to provide a fluid-tight seal between the first portion 518 of the lumen 516 and the second portion 519 of the lumen 516. As shown in FIG. 17, in some embodiments, the piston 544 can be disposed within the lumen 516 such that the piston 544 is a predetermined length 1 from an end portion (e.g., the first end portion 512) of the first section 510. In this manner, the volume of the first portion 518 of the lumen 516, and therefore the volume of the medicament 502 contained therein, can be adjusted, set, and or controlled to a predetermined amount.

In some embodiments, the method optionally includes coupling a cap to the first fitting such that the first portion of the lumen is fluidically isolated from a region outside of the first section of the tubular member, at 590. Referring to FIG. 17, the cap 538 is coupled to the first fitting 525. The cap 538 can be any suitable cap to fluidically isolate the first portion of the lumen. In this manner, the medicament 502 and/or the flow path of the medicament can remain sterile when the first section 510 of the tubular member 501 is not in use. For example, in some embodiments, the first section 510 of the tubular member 501 can be included in a medical kit similar to the kit 405 shown and described above. In some embodiments, for example, the cap 538 can be a frangible seal configured to be punctured and/or ruptured when a needle (not shown) is coupled to the first fitting 525.

Returning to the flow chart of FIG. 14, in some embodiments, the method optionally includes attaching a second fitting to a second end portion of the first section of the tubular member, at 592. As described above, the second fitting is configured to couple the second end portion of the first section to an actuator. Referring to FIG. 17, the second fitting 552 is coupled to the second end portion 511 of the first section 510 of the tubular member 501.

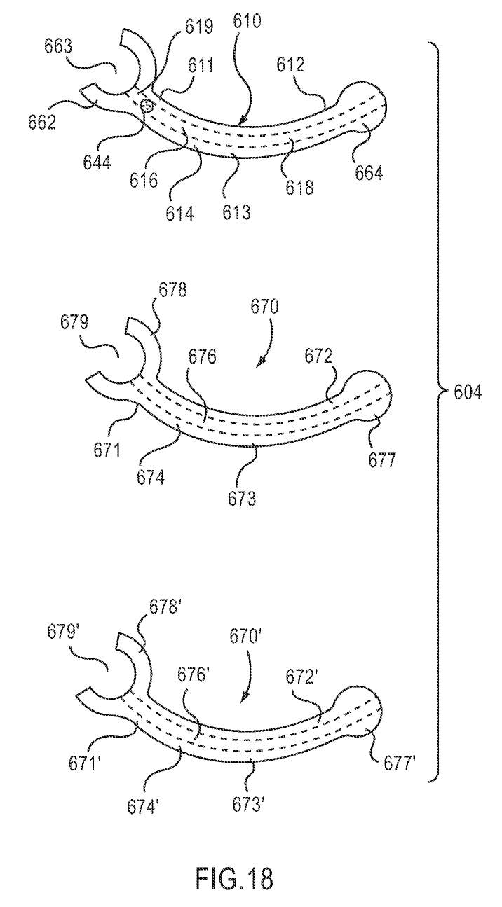

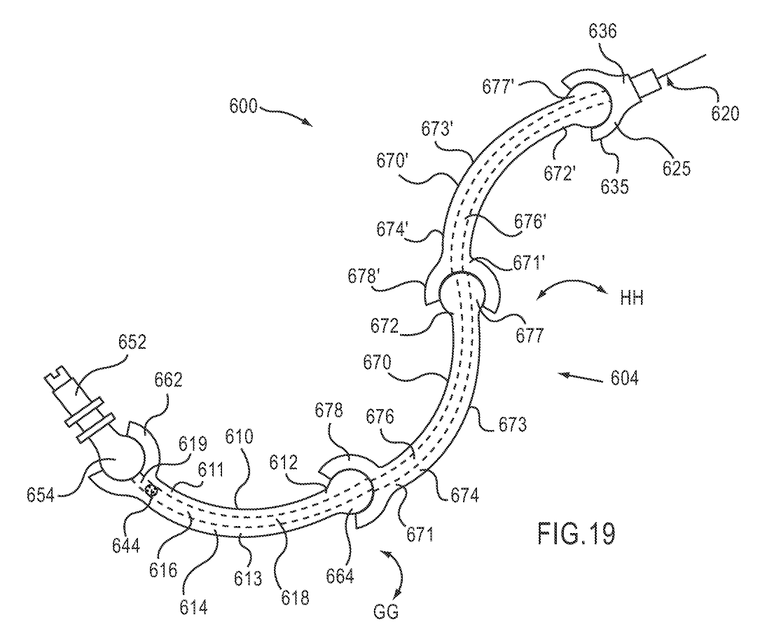

Although the tubular member 300 is shown and described above as being a monolithically-constructed flexible tube, in other embodiments, a flexible tubular member can be constructed from multiple components. Moreover, in some embodiments, a flexible tubular member can be constructed from multiple rigid components. Similarly stated, in some embodiments, a flexible tubular assembly can be constructed from multiple rigid tubular members. For example, FIGS. 18 and 19 are cross-sectional schematic illustrations of a tubular assembly 604 according to an embodiment in a first (unassembled) configuration and a second (assembled) configuration, respectively. The tubular assembly includes a first tubular member 610, a second tubular member 670 and a third tubular member 670′. The first tubular member 610, the second tubular member 670 and/or the third tubular member 670′ can be substantially inflexible (e.g., constructed from a rigid material). Similarly stated, the first tubular member 610, the second tubular member 670 and/or the third tubular member 670′ can be resistant to being deflected, displaced and/or deformed when an external load is applied thereto. As shown in FIG. 19, however, the first tubular member 610, the second tubular member 670 and the third tubular member 670′ can be coupled together to form the tubular assembly 604 that is flexible.

The first tubular member 610 has a side wall 614 that defines a lumen 616 therethrough. The first tubular member 610 includes a proximal end portion 611, a distal end portion 612, and a central portion 613 therebetween. At least the central portion 613 of the first tubular member 610 is curved, in a similar manner as described above. The proximal end portion 611 includes a coupling portion 662 that defines an opening 663 having a substantially spherical shape. As described in more detail below, the opening 663 of the coupling portion 662 can matingly receive a corresponding protrusion (e.g., protrusion 654) of a coupler (e.g., coupler 652) to removably couple the coupler to the proximal end portion 611 of the first tubular member 610. The distal end portion 612 of the first tubular member 610 includes a protrusion 664 having a substantially spherical shape. As described in more detail below, the protrusion 664 can be matingly received within a corresponding coupling portion (e.g., coupling portion 678) of another tubular member (e.g., the second tubular member 670) and/or a needle coupler (e.g., the needle coupler 625) to removably couple the distal end portion 612 of the first tubular member 610 to other components.

As described above, a piston 644 is disposed within the lumen 616 of the first tubular member 610 such that the tubular member 610 is divided into a first portion 618 and a second portion 619. A medicament, such as, for example a dermal filler, is disposed in the first portion 618 of the first tubular member 610. In this manner, the first portion 618 of the first tubular member 610 can function as a medicament container to contain the medicament. The length of the first tubular member 610, the diameter of the lumen 616 and/or the position within the lumen 616 at which the piston 644 is disposed can at any suitable value such that the first tubular member 610 can contain the desired volume of the medicament. For example, in some embodiment, the first tubular member 610 can contain approximately 0.5 cubic centimeters, 1 cubic centimeter, 2 cubic centimeters, 3 cubic centimeters, 5 cubic centimeters, 10 cubic centimeters, 15 cubic centimeters and/or 20 cubic centimeters of the medicament. In some embodiments, the first tubular member 310 can contain greater than 20 cubic centimeters of the medicament.

The second tubular member 670 has a side wall 674 that defines a lumen 676 therethrough. A medicament, such as, for example a dermal filler, is disposed within the lumen 676 of the second tubular member 670. The length of the second tubular member 670, and/or the diameter of the lumen 676 can at any suitable value such that the second tubular member 670 can contain the desired volume of the medicament. For example, in some embodiment, the second tubular member 670 can contain approximately 0.5 cubic centimeters, 1 cubic centimeter, 2 cubic centimeters, 3 cubic centimeters, 5 cubic centimeters, 10 cubic centimeters, 15 cubic centimeters and/or 20 cubic centimeters of the medicament. In some embodiments, the second tubular member 670 can contain greater than 20 cubic centimeters of the medicament. The openings of the lumen 676 can be covered by seals (not shown in FIGS. 18 and 19) such that the medicament remains within the lumen 676 in a sterile condition.

The second tubular member 670 includes a proximal end portion 671, a distal end portion 672, and a central portion 673 therebetween. At least the central portion 673 of the second tubular member 670 is curved, in a similar manner as described above. The proximal end portion 671 of the second tubular member 670 includes a coupling portion 678 that defines an opening 679 having a substantially spherical shape. The distal end portion 672 of the second tubular member 670 includes a protrusion 677 having a substantially spherical shape.

The third tubular member 670′ has a side wall 674′ that defines a lumen 676′ therethrough. A medicament, such as, for example a dermal filler, is disposed within the lumen 676′ of the third tubular member 670′. The length of the third tubular member 670′, and/or the diameter of the lumen 676′ can at any suitable value such that the third tubular member 670′ can contain the desired volume of the medicament. For example, in some embodiment, the third tubular member 670′ can contain approximately 0.5 cubic centimeters, 1 cubic centimeter, 2 cubic centimeters, 3 cubic centimeters, 5 cubic centimeters, 10 cubic centimeters, 15 cubic centimeters and/or 20 cubic centimeters of the medicament. In some embodiments, the third tubular member 670′ can contain greater than 20 cubic centimeters of the medicament. The openings of the lumen 676′ can be covered by seals (not shown in FIGS. 18 and 19) such that the medicament remains within the lumen 676′ in a sterile condition.

The third tubular member 670′ includes a proximal end portion 671′, a distal end portion 672′, and a central portion 673′ therebetween. At least the central portion 673′ of the third tubular member 670′ is curved, in a similar manner as described above. The proximal end portion 671′ of the third tubular member 670′ includes a coupling portion 678′ that defines an opening 679′ having a substantially spherical shape. The distal end portion 672′ of the third tubular member 670′ includes a protrusion 677′ having a substantially spherical shape.

As shown in FIG. 19, the first tubular member 610, the second tubular member 670 and the third tubular member 670′ can be coupled together to form the tubular assembly 604. More particularly, the protrusion 664 of the first tubular member 610 is matingly received within the coupling portion 678 of the second tubular member 670. In this manner, the first tubular member 610 can be coupled to the second tubular member 670 such that the lumen 616 of the first tubular member 610 can be placed in fluid communication with the lumen 676 of the second tubular member 670 (e.g., when a seal covering an opening of the first tubular member 610 and/or the second tubular member 670 is ruptured). The protrusion 664 of the first tubular member 610 and the coupling portion 678 of the second tubular member 670 form a substantially fluid-tight seal such the lumen 616 and the lumen 676 are fluidically isolated from a region outside of the first tubular member 610 and/or the second tubular member 670. In some embodiments, for example, the protrusion 664 of the first tubular member 610 and/or the coupling portion 678 of the second tubular member 670 can include a sealing member (e.g., an o-ring, a slip ring or the like) to form the substantially fluid-tight seal.

The protrusion 677 of the second tubular member 670 is matingly received within the coupling portion 678′ of the third tubular member 670′. In this manner, the second tubular member 670 can be coupled to the third tubular member 670′ such that the lumen 676 of the second tubular member 670 can be placed in fluid communication with the lumen 676′ of the third tubular member 670′ (e.g., when a seal covering an opening of the second tubular member 670 and/or the third tubular member 670′ is ruptured). The protrusion 677 of the second tubular member 670 and the coupling portion 678′ of the third tubular member 670′ form a substantially fluid-tight seal such the lumen 676 and the lumen 676′ are fluidically isolated from a region outside of the second tubular member 670 and/or the third tubular member 670′. In some embodiments, for example, the protrusion 677 of the second tubular member 670 and/or the coupling portion 678′ of the third tubular member 670′ can include a sealing member to form the substantially fluid-tight seal.

Moreover, as shown by the arrow GG in FIG. 19, when the first tubular member 610 is coupled to the second tubular member 670, the first tubular member 610 can be rotated relative to the second tubular member 670 via the spherical coupling between the first tubular member 610 and the second tubular member 670. Similarly, as shown by the arrow HH in FIG. 19, when the second tubular member 670 is coupled to the third tubular member 670′, the second tubular member 670 can be rotated relative to the third tubular member 670′ via the spherical coupling between the second tubular member 670 and the third tubular member 670′. In this manner, the first tubular member 610, the second tubular member 670 and the third tubular member 670′ can be coupled together to form the tubular assembly 604 that is flexible. Although the arrows GG and HH show rotation about a single axis, the spherical couplings described above allow rotation about any axis (i.e., three degrees of rotational freedom).

The tubular assembly 604 can be coupled to an actuator (not shown) and a needle assembly 620 to form a modular medical injector 600. More particularly, as shown in FIG. 19, the protrusion 677′ of the third tubular assembly 670′ can be matingly received within a first coupling portion 635 of a needle coupler 625. A second coupling portion 636 of the needle coupler can removably couple a needle assembly 620 to the needle coupler 625. The second coupling portion 636 can be, for example, a press-fit Luer fitting (e.g., a Luer-Slip™ fitting), a twist-on Luer fitting (e.g., a Luer-Lok™ fitting), a barbed Luer adapter and/or the like.

The opening 663 of the coupling portion 662 of the first tubular member 610 can matingly receive a corresponding protrusion of a coupler 652. The coupler 652 is configured to operatively couple an energy source (not shown in FIGS. 18 and 19) to the tubular assembly 604. The energy source can be any suitable source that produces a kinetic energy to move the piston 644 within the lumen 616 of the first tubular member 610, the lumen 676 of the second tubular member 670 and/or the lumen 676′ of the third tubular member 670′. In some embodiments an end portion of the lumen 616, an end portion of the lumen 676 and/or an end portion of the lumen 676′ can be flared to facilitate movement of the piston 644 therein. Similarly stated, in some embodiments, the diameter of the lumen 616, the diameter of the lumen 676 and/or the diameter of the lumen 676′ can be increased near the ends thereof to facilitate movement of the piston 644 therein.

The tubular members shown and described above can be constructed from any suitable material. For example, in some embodiments, a tubular member can be constructed from a rigid material, such as a rigid polymer, stainless steel, or the like. In other embodiments, a tubular member can be constructed from a flexible material. In yet other embodiments, a tubular member can be constructed from a material that is flexible and elastic, such that at least a portion of the tubular member can be flexible and deformable. In some embodiments, for example, a tubular member can be constructed from an organic polymeric composition, optionally an elastomer, for example a composition comprising a high strength polymer, for example a composition comprising a polyetheretherketone, a polyimide (e.g. Ultem), a polyurethane, a polysiloxane or a polyethersulfone.

In some embodiments, at least a portion of the tubular member can be transparent, translucent and/or opaque. In this manner, a user can visually observe the travel of the piston through the tubular member. Moreover, in some embodiments, a tubular member can include graduated markings to indicate the length and/or volume of a portion of the tubular member.

In some embodiments, a tubular member can include a reinforcing component to increase the transverse and/or longitudinal strength of the tubular member. In some embodiments, for example, a tubular member can be constructed of strands (e.g. fibers) that are optionally interlaced with each other, and placed within or outside a tube of polymeric material (for example a sleeve, e.g. a wire coil, surrounding or integral with a tube of polymeric material). The strands can be constructed from, for example, glass, a high-strength polymeric material, or a metal.

In some embodiments, a tubular member can include a coating and/or a liner on the inner surface that defines the lumen. The coating can, for example, improve the fluid-tight seal between the piston and the tubular member.

The pistons shown and described above can be constructed from any suitable material. For example, in some embodiments, a piston can be monolithically constructed from a single material. In other embodiments, a piston can be constructed from a combination of materials. In yet other embodiments, a piston can be constructed from multiple pieces (e.g., an inner core layer and an outer sealing layer). In some embodiments, for example, at least an outer surface of a piston can be constructed from a polymeric compositions having low surface energy, for example fluorinated polymers, e.g. polytetrafluoroethylene (PTFE), and/or which have a low coefficient of friction with the interior surface of the reservoir. In some embodiments, a piston can be constructed from an elastomeric compositions (e.g., silicone-based compositions). In some embodiments, a piston can be colored for easy visualization of piston movement within the tubular member.

The medicaments and/or dermal fillers described above can be any material suitable for augmenting soft tissue. In some embodiments, a medicament and/or dermal filler can include a pain reliever, such as, for example, lidocaine. In other embodiments, a medicament and/or dermal filler can include a colorant and/or a marker. For example, in some embodiments a medicament and/or dermal filler can include a radio-opaque marker. In other embodiments, a medicament and/or dermal filler can include a tattoo ink.

In some embodiments, the medicament can be a fluid that is characterized by a substantially linear shear stress as a function of the rate of shear strain applied thereto. Said another way, in some embodiments, the medicament can be a Newtonian fluid having a viscosity that varies substantially only as a function of its temperature and pressure. In other embodiments, the medicament can be a fluid that is characterized by a non-linear shear stress as a function of the rate of shear strain applied thereto. Said another way, in some embodiments, the medicament can be a non-Newtonian fluid having a viscosity that varies according other factors, such as, for example, the magnitude of and/or rate of increase of a force applied to the medicament.

In some embodiments, a medicament and/or a dermal filler as described above can include, for example, a side chain crystalline (SCC) polymer of the type disclosed in International Patent Application No. PCT/US2007/023226, entitled “Compositions, Devices and Methods for Modifying Soft Tissue,” which is incorporated herein by reference in its entirety. In other embodiments, a dermal filler can include hyaluronic acid. In yet other embodiments, a dermal filler can include polyacrylamide, collagen (either human and/or bovine), polymethylmethacrylate, silicone, calcium hydroxylapatite (CaHA), hydrophilic polyacrylamid gel (PAAG), and/or poly-L-lactic acid hydrogel (PLLA).

In some embodiments, a medicament and/or a dermal filler can include any of the following commercially-available dermal fillers: Puragen™ and its derivatives, produced by Mentor Corporation, Belotero® and its derivatives, produced by Merz Pharmaceuticals, BIO-ALCAMID™ and its derivatives, produced by Polymekon S.R.L., Outline® and its derivatives, produced by ProCytech, HylaNew® and its derivatives, produced by Prollenium Medical Technologies, Inc., Restylane® and its derivatives, produced by Q-Med or Medicis Pharmaceutical Corporation, Reviderm USA and its derivatives, produced by Rofil Medical International N.V., Teosyal® and its derivatives, produced by Teoxane Laboratories, Fascian® and its derivatives, produced by Fascia Biosystems, LLC, FG-5017 and its derivatives, produced by Fibrogen, Inc., Amazingel and its derivatives, produced by FuHua High Molecular Matter Company, Ltd., Laresse® Dermal Filler and its derivatives, produced by FzioMed, Inc., Zyderm® and its derivatives, produced by Inamed Corporation, Isolagen® and its derivatives, produced by Isolagen, Inc., MacDermol® and its derivatives, produced by Laboratories ORGéV, Juvéderm® and its derivatives, produced by L.E.A. Derm, Hyaluderm® and its derivatives, produced by LCA Pharmaceutical, Silikon® 1000 and its derivatives, produced by Alcon, Inc., Esthélis and its derivatives, produced by Antesis® S.A., Artefill® and its derivatives, produced by Artes Medical, Inc., Radiesse® and its derivatives, produced by BioForm Medical, Inc., Matridex® and its derivatives, produced by BioPolymer GmbH & Co. KG, Evolence® and its derivatives, produced by ColBar LifeScience Ltd., Aquamid® and its derivatives, produced by Contura International A/S, SurgiDerm® and its derivatives, produced by Labortoire Corneal® Development, Rhegecoll and its derivatives, produced by Dermabiol Institute of Kuhra Vital GmbH, DermaLive® and its derivatives, produced by Derma Tech, and/or Sculptra™ and its derivatives, produced by Dermik® Laboratories.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, although the flow chart of the method 580 is shown as including filling a tubular member, at 582, before cutting the tubular member, at 584, in other embodiments, a method can include cutting a tubular member before filling the tubular member with a medicament.

Although the tubular member 210 is shown and described above as having a curved central portion 213 in both the first configuration and the second configuration, in other embodiments, a tubular member of the types shown and described above can be substantially linear in the first configuration and/or the second configuration. For example, in some embodiments, a rigid tubular member, such as tubular member 610 can be substantially linear.

Although the tubular member 210 is shown as having a first configuration and a second configuration, in other embodiments, a tubular member can have any number of different configurations and/or shapes. For example, in some embodiments, a flexible tubular member can have a first configuration, in which the flexible tubular member is coiled through more than one revolution, a second configuration, in which the flexible tubular member is curved, and a third configuration in the flexible tubular member is substantially linear. Moreover, in such embodiments, the flexible tubular member can have any number of configurations and/or shapes between the first configuration, the second configuration and/or the third configuration.

Although the piston 344 is shown and described above as including a sealing portion 345 that forms a fluid tight seal with the inner surface 315 of the tubular member 310, in other embodiments, a piston need not form a fluid tight seal within the lumen of a tubular member. For example, in some embodiments, the medicament in the first portion of the tubular member and a pressurized fluid in the second portion of the tubular member can be immiscible. Thus, in the absence of a fluid-tight seal between the first portion of the tubular member and the second portion of the tubular member, the medicament and the pressurized fluid do not readily mix.

Although the medicament containers are shown and described above as including a piston, in other embodiments, a medicament container can be devoid of a piston. For example, in some embodiments, a medical injector can include a tubular member devoid of a piston that is configured to be coupled to source of pressurized fluid. A dermal filler contained within the tubular member can have a high viscosity such that it will not readily mix with the pressurized fluid. Accordingly, to actuate the medical injector, the pressurized fluid is conveyed into the tubular member and into direct contact with the dermal filler to be injected, thereby moving the dermal filler within the tubular member.

Although the tubular members disclosed herein include a lumen that is generally described as having a circular cross-sectional shape, in some embodiments, a tubular member can include a lumen having a non-circular cross-sectional shape. For example, in some embodiments, a tubular member can include a lumen having a substantially elliptical cross-sectional shape. In other embodiments, a tubular member can include a lumen having a substantially triangular cross-sectional shape.

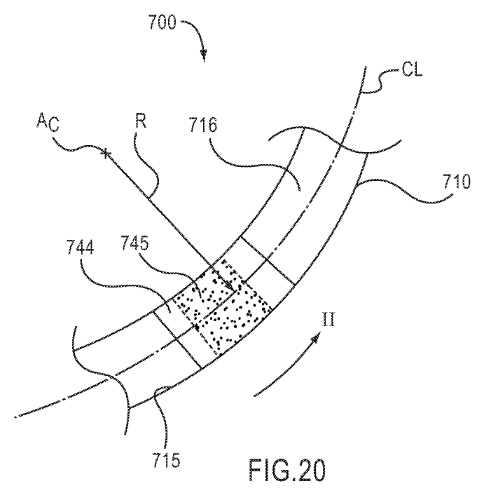

Although the piston 144 is shown and described above as having a spherical shape, in other embodiments, a piston can have any suitable shape. For example, in some embodiments, a piston can have an oblong (e.g., oval) shape. In other embodiments, a piston can have a diamond shape (when viewed from the side). In yet other embodiments, a piston can have a substantially cylindrical shape. For example, FIG. 20 is a schematic illustration of a portion of a medical injector 700 according to an embodiment. The medical injector 700 includes a tubular member 710 and a piston 744. The tubular member 710 defines a lumen 716 therethrough having a longitudinal center line CL. For clarity, the tubular member 710 is shown as being clear so that the piston 744 disposed therein can be shown. As shown in FIG. 20, at least a portion of the tubular member 710 is curved. More specifically the portion of the tubular member 710 is curved about an axis of curvature A_{C} that is substantially normal to the center line CL. Thus, at least a portion of the center line CL has a radius of curvature R about the axis of curvature A_{C}.

The piston 744 is disposed within the lumen 716 of the tubular member 710 such that the tubular member 710 is divided into a first portion and a second portion. The first portion of the tubular member 710 can contain a medicament, as described above. The piston 744 can move within the lumen 716 along the center line CL. When the piston 744 moves within the tubular member 710, as shown by the arrow II in FIG. 20, the medicament is conveyed from the first portion of the tubular member 710, as described above.

As shown in FIG. 20, the piston 744 has a nominally cylindrical shape, and includes a sealing portion 745 (shown as a shaded region in FIG. 20) that contacts the inner surface 715 of the tubular member 710 when the piston 744 is disposed within the lumen 716. Additionally, the piston 744 is flexible such that the sealing portion 745 of the piston 744 can conform to the inner surface 715 of the tubular member 710 when the piston 744 moves within the lumen 716. More particularly, the piston 744 can bend about the axis of curvature A_{C} when the piston 744 moves within the lumen 716. In this manner, the shape of the piston 744 can change shape from a nominally cylindrical shape to a curved shape to substantially match the shape of the tubular member 710.

The sealing portion 745 of the piston 744 has a length dimension and a width dimension, and therefore contacts the inner surface 715 of the tubular member 710 along a two-dimensional area. Although the sealing portion 745 is shown as having a length less than the length of the piston 744, in other embodiments, the sealing portion 745 can extend the entire length of the piston 745. Although the sealing portion 745 contacts the inner surface 715 along a two-dimensional area, because the piston 744 is constructed from a flexible material, the piston 744 can move within the lumen 716 through tight bends. In some embodiments, for example, the piston 744 can move within the lumen 716 along the center line CL through a curved portion having a radius of curvature R that is as low as twice the diameter of the lumen 716

Although the piston 244 is shown and described above as having an oblong shape, in other embodiments, a piston can have any suitable shape. For example, in some embodiments, a piston can have a substantially cylindrical shape. In other embodiments, an end surface of a piston can be substantially flat. In yet other embodiments, an end surface of a piston can be concave or convex. Moreover, as described above, in some embodiments, the piston 244 can be flexible.

Although the side wall 314″ of the tubular member 310″ (see e.g., FIG. 9) is shown and described as being elastically deformable, in other embodiments, a side wall of a tubular member can be rigid such that the side wall is not readily deformable. For example, in some embodiments, a tubular member can include a rigid side wall defining a lumen having a first diameter. A piston having a second diameter greater than the first diameter can be disposed within the lumen. The piston can be configured to elastically deform in the region where the inner surface of the side wall contacts the piston. In this manner, the piston can change shape to form a fluid-tight seal within the lumen of the tubular member.

Although the sleeve 403 is shown as producing a shoulder 417 within the lumen 416, in other embodiments, the sleeve 403 can limit the movement of the piston 444 without producing a shoulder 417 within the lumen 416. Although the sleeve 403 is shown as causing the diameter d2 of the lumen to be reduced to a value less than the nominal diameter d3, in other embodiments, the sleeve 403 need not cause the diameter of the lumen 416 to be reduced. For example, in some embodiments, a sleeve can be constructed from a rigid material, and can therefore, limit the movement of the piston therein by preventing a portion of the tubular member from deforming.

Although various embodiments have been described as having particular features and/or combinations of components, other embodiments are possible having a combination of any features and/or components from any of embodiments where appropriate. For example, in some embodiments, a medical injector can include multiple tubular members coupled together similar to the medical injector 600 shown and described above with reference FIGS. 18 and 19, and at least one of the tubular members can be flexible similar to the tubular member 300 shown and described above with reference to FIGS. 5 and 6.

What is claimed is:

1. An apparatus, comprising:
a tubular member defining a lumen, a distal end portion of the tubular member configured to be coupled to a needle, at least a central portion of the tubular member being curved; and
a spherical piston movably disposed with the lumen such that the lumen of the tubular member is divided into a first portion and a second portion, the first portion of the lumen configured to contain a medicament.

2. The apparatus of claim 1, wherein at least the central portion of the tubular member is flexible.

3. The apparatus of claim 1, wherein the spherical piston is configured to contact an inner surface of the tubular member to form a substantially fluid-tight seal between the first portion of the lumen and the second portion of the lumen.

4. The apparatus of claim 1, wherein a diameter of the spherical piston is greater than a diameter of the lumen.

5. The apparatus of claim 1, wherein the proximal end portion of the tubular member is configured to be coupled to an energy source that produces a kinetic energy to move the spherical piston within the lumen of the tubular member such that the medicament can be conveyed from the first portion of the lumen through the needle.

6. The apparatus of claim 1, wherein the proximal end portion of the tubular member includes a coupler configured to couple the proximal end portion of the tubular member to a source of pressurized fluid such that a fluid-tight seal is formed between the source of pressurized fluid and the second portion of the lumen.

7. The apparatus of claim 1, wherein the distal end portion of the tubular member includes a coupler configured to removably couple the distal end portion of the tubular member to the needle.

8. The apparatus of claim 1, wherein the spherical piston is disposed within the lumen such that a length of the first portion of the lumen is at least 0.5 meters.

9. An apparatus, comprising:
a tubular member defining a lumen, a distal end portion of the tubular member configured to be coupled to a needle, at least a central portion of the tubular member being flexible; and
a piston movably disposed with the lumen such that the lumen of the tubular member is divided into a first portion and a second portion, the first portion of the lumen configured to contain a medicament, the piston configured to contact an inner surface of the tubular member solely along a locus of points.

10. The apparatus of claim 9, wherein the piston and the inner surface of the tubular member collectively form a substantially fluid-tight seal between the first portion of the lumen and the second portion of the lumen.

11. The apparatus of claim 9, wherein the piston is a sphere.

12. The apparatus of claim 9, wherein the proximal end portion of the tubular member includes a coupler configured to couple the proximal end portion of the tubular member to a source of pressurized fluid such that a fluid-tight seal is formed between the source of pressurized fluid and the second portion of the lumen.

13. The apparatus of claim 9, wherein the spherical piston is disposed within the lumen such that a volume of the first portion of the lumen is at least 1 cubic centimeter.

14. The apparatus of claim 9, further comprising a controller coupled to the distal end portion of the tubular member, the controller configured to be grasped by a user, the controller configured to limit a flow of the medicament from the first portion of the lumen through the needle.

15. The apparatus of claim 9, wherein at least the central portion of the tubular member is configured to be coiled about an axis substantially normal to a center line of the tubular member through at least one revolution.

16. The apparatus of claim 9, wherein at least the central portion of the tubular member is configured to be coiled about an axis substantially normal to a center line of the tubular member such that a radius of curvature of the central portion is less than approximately 10 centimeters.

17. A method, comprising:
filling a tubular member with a medicament; and
cutting the tubular member into a first section and a second section, at least the first section being filled with the medicament, the first section of the tubular member having a predetermined length.

18. The method of claim 17, wherein the medicament is a dermal filler having a nominal a viscosity of at least 1000 centipoise.

19. The method of claim 17, wherein the predetermined length of the first section is at least 0.5 meters.

20. The method of claim 17, further comprising:
attaching a fitting to an end portion of the first section, the fitting configured to removably couple the end portion of the first section to a needle.

21. The method of claim 17, further comprising:
attaching a first fitting to a first end portion of the first section, the first fitting configured to removably couple the first end portion of the first section to a needle; and
attaching a second fitting to a second end portion of the first section, the second fitting configured to removably couple the second end portion of the first section to an actuator.

22. The method of claim 17, further comprising:
disposing, after the cutting, a piston within a lumen defined by the first section of the tubular member.

23. The method of claim 17, further comprising:
attaching a fitting to a first end portion of the first section, the fitting configured to removably couple the first end portion of the first section to a needle;
disposing a piston within a lumen defined by the first section of the tubular member, such that the lumen of the first section of the tubular member is divided into a first portion and a second portion, at least the first portion of the lumen containing the medicament; and
coupling a cap to the fitting such that the first portion of the lumen is fluidically isolated from a region outside of the first section of the tubular member.

24. The method of claim 17, wherein the predetermined length of the first section is such that a volume of the medicament within the first section is at least 1 cubic centimeter.
